(19) United States
(12) Patent Application Publication
(10) Pub. No.: US 2009/0093493 A1
(43) Pub. Date: Apr. 9, 2009

(54) 1-PHENYLALCOXY-2-BETA-PHENYLETHYL DERIVATIVES AS P-GLYCOPROTEIN (P-GP) INHIBITORS USEFUL IN DRUG RESISTANCE EVENTS

(76) Inventors: Francesco Berardi, Noicattaro BA (IT); Nicola Antonio Colabufo, Triggiano BA (IT); Roberto Perrone, Bari BA (IT); Aldo Balsamo, S. Concordio LU (IT); Simona Rapposelli, S. Concordio LU (IT); Maria Digiacomo, Pisa PI (IT)

Correspondence Address: Joseph Fischer, Esquire;Beusse Wolter Sanks Mora & Maire, P.A., 390 North Orange Avenue, Suite 2500, Orlando, FL, 32801, US

(21) Appl. No.: 11/869,136

(22) Filed: Oct. 9, 2007

(51) Int. Cl. A61K 31/497; C07D 217/00; C07D 211/04; C07D 401/04; C07D 241/04; A61K 31/47; A61K 31/445; A61P 35/00

(52) U.S. Cl. 514/252.14; 546/139; 546/184; 544/333; 544/358; 514/315; 514/307

(57) ABSTRACT

The invention relates to a new class of compounds, which are 1-phenylalcoxy-2-β-phenylethyl derivatives, as P-glycoprotein (P-GP) inhibitors. These compounds are useful in drug resistance events. They have been shown able to inhibit in a dose-dependent manner Glycoprotein-P (P-gp) activity in cell lines in which the expression of said glycoprotein is very high, like Caco-2 (human colon cancer) cells and MCF7/Adr (adriamycin-resistant human breast carcinoma) cells. The invention also relates to methods of production and the utilization of such compounds as medicaments useful in the treatment of states linked to the difficulty for some drugs to cross the blood-brain barrier (BBB) and generally within the context of the problems of drug resistance induced by chemotherapy agents.

### TECHNICAL FIELD OF THE INVENTION

The invention relates to a new class of compounds able to inhibit in a dose-dependent manner Glycoprotein-P (P-gp) activity in cell lines in which the expression of said glycoprotein is very high, like Caco-2 (human colon cancer) cells and MCF7/Adr (adriamycin-resistant human breast carcinoma) cells. The invention also relates to the utilization of such compounds as medicaments useful in the treatment of states linked to the difficulty for some drugs to cross the blood-brain barrier (BBB) and generally within the context of the problems of drug resistance induced by chemotherapy agents.

### STATE OF THE PRIOR ART

P-glycoprotein is an ATP-ase-type extrusion pump located on the cell membrane and able to actively transport various types of molecules across the membrane. Tumor cell lines, like Caco-2 (human colon cancer) cells and MCF7/Adr (adriamycin-resistant human breast carcinoma) cells, are known in which the expression of said glycoprotein is very high. P-gp stability and location have been studied by Zhang et al. 2004, Mol Pharmacol. 66, 395-403).

Two binding sites of P-gp transported molecules are thought to exist: the R site, or rhodamine site, and the H site or Hoechst 33342 site. Various substances are known which are able to interact with such complex binding sites by influencing P-gp function. Interaction between these compounds and the sites has been studied by Lugo et al. 2005, Biochemistry, 44, 14020-14029; and by Sharom et al. 2002 Biochemistry, 41, 4744-4752. Moreover, on the basis of these preliminary data there have been conducted the first structure-activity relationship studies for substrates behaving as P-gp inhibitors (Wang et al. 2003, J. Clin. Pharm. Ther. 28, 203-228).

In the last few years, new putative compounds having P-gp inhibition activity have been developed, among them isoxazolic tricyclic derivatives (Norman et al. 2005, Bioorg. Med. Chem. Lett. 15, 5526-5530), analogs of pervilleine and of verapamil (Teodori et al. 2005, J. Med. Chem. 48, 7426-7436), baicalein-analog flavonoid derivatives (Lee et al. 2004, J. Med. Chem. 47, 1413-1422), quinoxalinone derivatives (Lawrence et al. 2001, J. Med. Chem., 44, 594-601). Results in the abovementioned works, though interesting, are not comparable since they come from different protocols. Moreover, and this is the lacking aspect in the biological evaluation performed, no intrinsic cytotoxicity assays are reported for the compounds. Accordingly, no comparison is possible of these compounds with the sole ascertained pure inhibitor reported in literature, GF120918 (Elacridar) described hereinafter.

Lastly, Taub et al. 2005, Drug Metab. Dispos. 33, 1679-1687 and Polli et al. 2001, JPET, 299, 620-628 describe the assessment methodology for compounds interfering with P-gp activity, though in different manners (substrate, inhibitor, inducer, non-transported substrate). In the most significant works relating to P-gp modulation, even before entering the biological assessment of the modulating compounds, it is highlighted the importance of the selection of the biological assay, the cell line overexpressing the glycoprotein P (Caco-2, MDCK-MDR1 and MDCK-WT) and the relevance of the concentration of the P-gp modulating compound to be used in the experiment (Taub et al. supra).

Over the last few years there have been singled out by Polli et al. 2005 JPET299, 620-628 pharmacological assays that allowed to characterize and classify the compounds exerting an interference with P-gp activity. These compounds have been classified according to the following scheme:

| Category I | Category II (substrates) |
|---|---|
| a) non-substrates | a) non-transported substrates |
| b) substrates | b) transported substrates |
| c) inhibitors | |

Under said subdivision, to date the sole pure inhibitor of P-gp reported in literature is GF120918 (Elacridar) in Phase I.

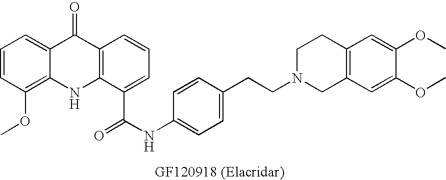

All other compounds reported to date in the literature either are no pure inhibitors under the above-reported classification, or their pharmacological characterization concerning the specific assays is incomplete.

To date, the best P-gp inhibitors entail drawbacks preventing their therapeutic use in association to chemotherapy agents in the treatment of neoplasias that have become resistant to chemotherapy treatment, as these compounds exert P-gp inhibition at high doses only, with the consequent onset of serious side effects. Verapamil, e.g., is a P-gp inhibitor only at high doses (200 μM), whereas at low doses it is a substrate. In addition, at doses at which it acts as inhibitor it equally acts as a Ca++ ion antagonist on L-type channels. Moreover, under said classification verapamil belongs to Category IIA (non-transported substrates).

Analogous considerations can be made for cyclosporine-A, which acts as P-gp inhibitor only at high doses, whereas at low concentrations it behaves as transported substrate (Category IIB). These two examples better focus the problems: on the one hand the lack of pure P-gp inhibitors, on the other hand the P-gp inhibition effect that may be attained, at certain concentrations, even with compounds that are not pure inhibitors (Verapamil, Cyclosporine-A).

Notwithstanding the above, there remains a need in the art for molecules able to interfere with P-gp activity, in particular molecules having inhibition activity.

### BRIEF DESCRIPTION OF THE FIGURES

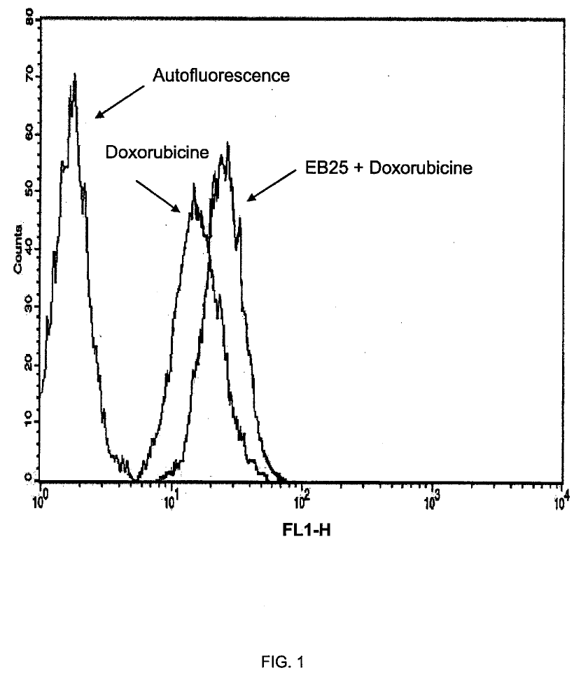

FIG. 1: The Figure illustrates cytofluorimetry results for doxorubicin on MCF/Adr in the presence and in the absence of the P-gp inhibitor compound (EB25). In particular, verapamil was included as reference compound having P-gp inhibition activity: in FIG. 1 there are highlighted the curves corresponding to:
autofluorescence,
Doxorubicin in the absence of compounds,
Doxorubicin+3-{2-[2-(3-methoxyphenyl)ethyl]phenoxy}propan-1-[4-(2-pyrimidyl)piperazine] (EB25);

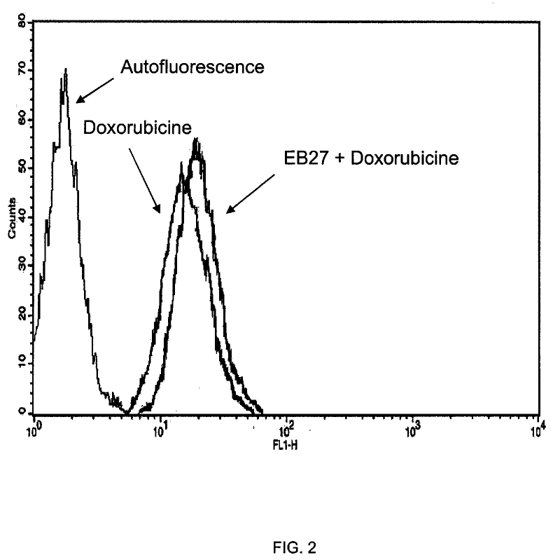

FIG. 2: The Figure illustrates cytofluorometric results for doxorubicin on MCF/Adr in the presence and in the absence of the P-gp inhibitor compound EB27. There are indicated the curves corresponding to:
autofluorescence.
Doxorubicin in the absence of compounds,
Doxorubicin+1-[(3-methoxybenzyl)oxy]-2-[2-(3-methoxyphenyl)ethyl]benzene (EB27).

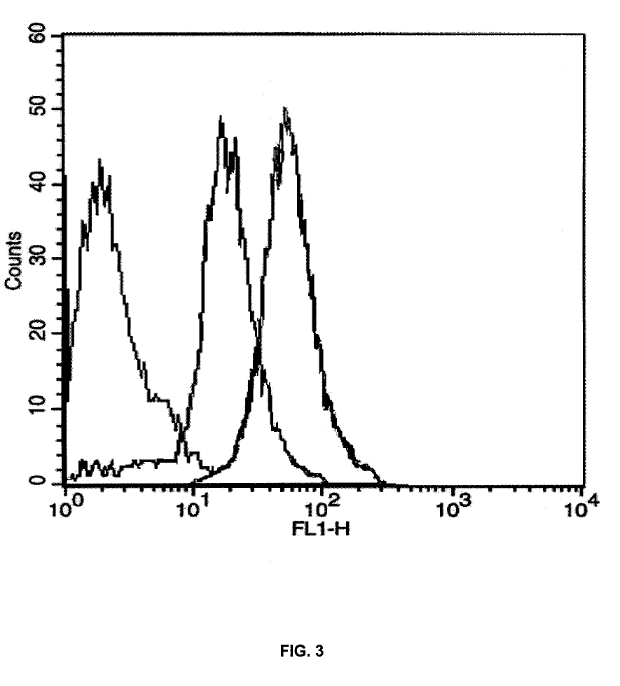

FIG. 3: The Figure illustrates cytofluorometric results for doxorubicin on MCF/Adr in the presence and in the absence of the P-gp inhibitor compound EB12. There are indicated the curves corresponding to:
autofluorescence (black).
Doxorubicin in the absence of compounds (dark grey),
Doxorubicin+2({2-[2-(3-methoxyphenyl)ethyl]phenoxy}methyl]pyridine (EB12) (light grey).

### DETAILED DESCRIPTION OF EMBODIMENTS OF THE INVENTION

The scope of the present invention includes providing novel molecules able to interfere with P-gp activity, in particular novel molecules having pure inhibition activity.

The present invention is based on the identification of a novel family of molecules able to variously interfere with P-gp activity as inhibitors or as transported or non-transported substrates. Therefore, these substances find application directly in the inhibition of the ATP-ase extrusion pump activity of P-gp or as modulating agents thereof, or again as starting products for the research and development of novel effective inhibitor molecules.

Hence, the present invention includes a family of compounds having the general formula as indicated in claim 1.

A second aspect of the present invention includes methods of preparation of compounds of the noted family of compounds.

A third aspect of the present invention regards therapeutic treatments of oncological pathologies or central nervous system related pathologies involving P-glycoprotein function, comprising administering a therapeutically effective amount of a compound of the invention, and preferably further comprising administering an anti-tumor medicament or a medicament active on the central nervous system.

A fourth aspect of the invention regards a pharmaceutical composition comprising a therapeutically effective amount of the compounds of the invention and a pharmacologically acceptable excipient.

A fifth aspect of the invention pertains to associations of the compounds according to the invention with anti-tumor medicaments or medicaments active on the central nervous system.

A further aspect of the invention are pharmaceutical kits containing compounds according to the invention with anti-tumor medicaments or medicaments active on the central nervous system.

The above and other aspects of the present invention will be made evident in the light of the following.

Compounds of the Invention

The invention relates to a family of novel compounds having the following general formula:

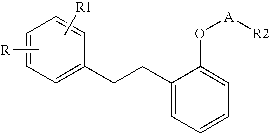

where:

R and R1 may independently be hydrogen atoms, an alkyl (methyl, ethyl, propyl, isopropyl, butyl, isobutyl or tertbutyl) group, an alkoxy (methoxy, ethoxy, n-, iso-propyloxy) group, a halogen (F, Cl, Br I) atom, a cyano group, a thiol group, a hydroxyl group, a (C1, C2, C3) thioalkyl group;

R2 may be a 5- or 6-member aryl group, unsubstituted or substituted with a (C1, C2, C3) alkyl group, with a (C1, C2 or C3) alkoxy group or with halogen atoms (Cl, Br, F); or a 5- or 6-member aromatic or aliphatic heterocyclic group, containing one or more atoms of nitrogen or oxygen or sulphur, even N-alkyl substituted or N-aryl substituted, N-halogenaryl substituted or N-alkyl-aryl substituted or N-alkyl-halogenaryl substituted, like N-alkylpiperazine, N-phenylpiperazine, N-benzylpiperazine, N-2-,3-,4-alcoxybenzyl-piperazine, N-pyrimidylpiperazine, N-1-,2-,3-pyridylpiperazine, an N-benzyl substituted or N-picolyl substituted 3-piperidine or suitably substituted aminoalkylaryls, like aminoethyl-pyridine, aminomethylpyridine, aminoethylpyrrole, benzylamine and substituted analogs.

R2 may also be an —NR3R4-type group, where R3 and R4 may independently be a hydrogen atom, alkyl (methyl, ethyl, propyl, isopropyl, butyl, isobutyl, tertbutyl, pentyl) groups, (C1-C5) alkyl groups substituted with 5- or 6-member aromatic or heteroaromatic systems, or (C1-C5) alkyl chains bound to the nitrogen to form a 5- or 6-member aliphatic heterocycle condensed or bound to a further aromatic or heteroaromatic group, it also 5- or 6-member, and A represents a linear or branched C1, C2, C3 or C4 alkyl chain, or a single or repeating chain of formula —CH₂—CH(OH)CH₂—.

A specific subgroup of the general formula indicated above comprises molecules wherein A is a —CH₂— group and R₂ is a phenyl group, optionally substituted with halogen atoms, with alkyl groups or with alkoxyl groups or a pyridine (2-, 3- or 4-pyridyl) group, it also optionally substituted.

A second subgroup comprises molecules wherein A is a linear —(CH₂)₂—, —(CH₂)₃—, —(CH₂)₄— chain and R₂ comprises a piperazine and piperidine group optionally substituted or condensed with other nitrogenated heterocycle, e.g. substituted with a pyrimidine, pyrazine or pyridazine group or with aromatic clusters optionally substituted with alkyl, alkoxyl or halogen groups.

A third subgroup comprises molecules wherein A is a linear —CH₂—CHOH—CH₂— chain.

Specific examples of compounds of the invention are:

1-[(3-methoxybenzyl)oxy]-2-[2-(3-methoxyphenyl)ethyl]benzene (EB27);

2({2-[2-(3-methoxyphenyl)ethyl]phenoxy}methyl]pyridine (EB12);

3-{2-[2-(3-methoxyphenyl)ethyl]phenoxy}propan-1-[4-(2-pyrimidyl)piperazine] (EB25);

3-({2-[2-(3-methoxyphenyl)ethyl]phenoxy}methyl)pyridine (EB18);

1-(benzyloxy)-2-(2-phenylethyl)benzene (EB 30), as well as all other compounds reported in the table below.

| Com- | | | |
|---|---|---|---|
| pound | R | R₁ | A-R₂ |
| EB27 | H | OCH₃ | 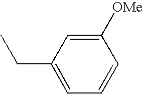 |
| EB12 | H | OCH₃ | 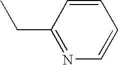 |
| EB18 | H | OCH₃ | 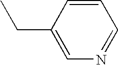 |
| EB30 | H | H | 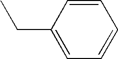 |
| EB28 | H | OCH₃ | 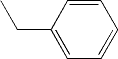 |
| EB29 | H | H | 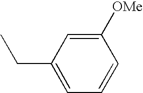 |
| EB13 | H | H | 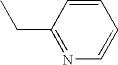 |
| EB14 | H | H | 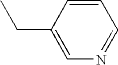 |
| EB25 | H | OCH₃ | 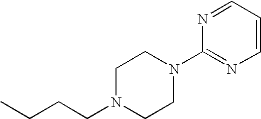 |
| EC43 | H | OCH₃ | 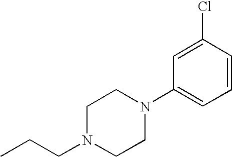 |
| EC77 | H | OCH₃ | 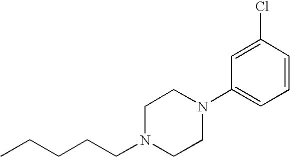 |
| EC52 | H | OCH₃ | 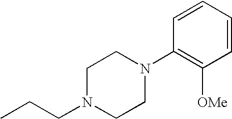 |
| EC65 | H | OCH₃ | 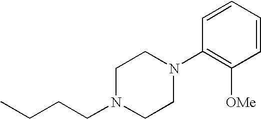 |
| EC74 | H | OCH₃ | 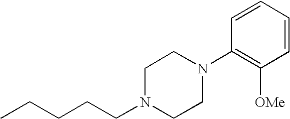 |
| EC57 | H | OCH₃ | 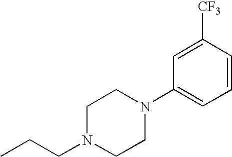 |
| EC68 | H | OCH₃ | 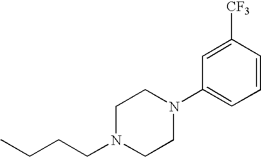 |
| EC76 | H | OCH₃ | 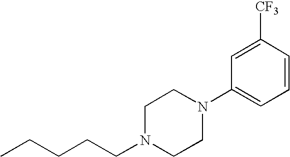 |
| MC71 | H | OCH₃ | 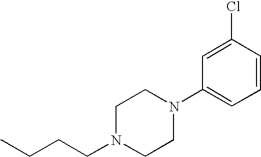 |
| EC66 | H | OCH₃ | 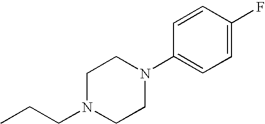 |
| EC67 | H | OCH₃ | 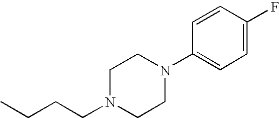 |
| EC78 | H | OCH₃ | 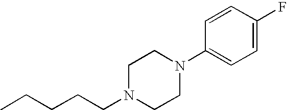 |
| EC60 | H | OCH₃ | 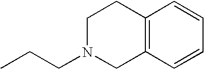 |
| EC75 | H | OCH₃ | 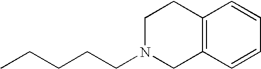 |
| MC70 | H | OCH₃ | 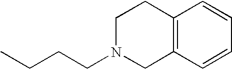 |
| MC50 | H | OCH₃ | 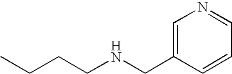 |
| EB17 | H | OCH₃ | 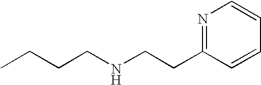 |
| MC72 | H | OCH₃ | 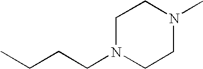 |
| R28 | H | OCH₃ | 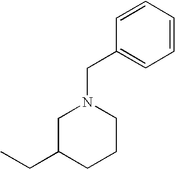 |
| MB33 | H | OCH₃ | 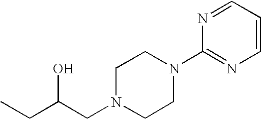 |
| MB31 | H | OCH₃ | 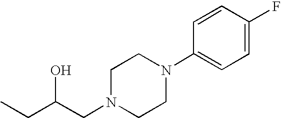 |
| MB32 | H | OCH₃ | 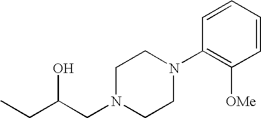 |
| MC74 | H | OCH₃ | 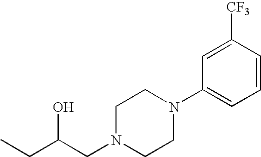 |
| MC67 | H | OCH₃ | 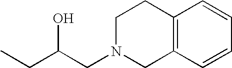 |

Biological Activity

The compounds of the invention variously interfere with P-gp activity. Therefore, these substances find application directly in the inhibition of the ATP-ase extrusion pump activity of P-gp or as modulating agents thereof, or again as starting products for the research and development of novel effective inhibiting molecules.

The activity exerted by the molecules of the invention is estimated, in accordance with the method described by Taub et al. (supra) or by Polli et al (supra), using as standard known and freely available cell lines able to overexpress P-gp, such as CACO-2, CF7/Adr, MDCK-MDR1 or MDCK-WT.

Biological Assays

The biological assays performed verify a) transport for each for each candidate compound by P-gp (apparent permeability determined in Caco-2 cells); b) ATP-ase activation in Caco-2 cells; c) inhibition of [³H] Vinblastine basolateral-apical transport (in Caco-2 cells) and of rhodamine (in guinea pig ileum).

To perform the assay, cell lines characterized by high P-gp expression (e.g. Caco-2) are seeded on 96-well plates (Millipore patent) and cultivated to confluence, affording a tight cell monolayer. Vinblastine, a known chemotherapy agent, is one of the best substrates for the (P-gp) extrusion pump. The cell monolayer is contacted with a solution of [3H]vinblastine, evaluating the ability of the ATP-ase pump to expel the cell-permeated compound through the lipid bilayer of the cell membrane. At equilibrium, the amount of [³H]vinblastine, both permeated and non-permeated into the cell, is measured in the absence and in the presence of one of the compounds of the invention. In the presence of inhibiting compounds, the concentration of [3H]vinblastine remaining inside the cell following P-gp blocking increases with the increasing of the concentration of the compound under examination. Moreover, cell ATP depletion is evaluated in the presence and in the absence of the same compounds. The results obtained highlight how to an inhibiting effect exerted by the compounds under examination there corresponds a decrease of the cell ATP-ase activity that can be ascribed to P-gp pump blocking.

Another assay is performed on MCF-7/ADr cells. These cells, resistant to the chemotherapy agent doxorubicin, are exposed for some hours to the compound under examination and subsequently treated with doxorubicin. The amount of doxorubicin entering the cell following treatment with the compounds under examination is then measured.

The above-described assays highlighted that molecules like 1-[(3-methoxybenzyl)oxy]-2-[2-(3-methoxyphenyl)ethyl]benzene (EB27), 2({2-[2-(3-methoxyphenyl)ethyl]phenoxy}methyl]pyridine (EB12), and 3-{2-[2-(3-methoxyphenyl)ethyl]phenoxy}propan-1-[4-(2-pyrimidyl)piperazine] (EB25) are potent inhibitors classifiable to Category IC and highlight a very high endocellular accumulation. These substances display inhibition activity already at nanomolar doses, i.e. from 5 to 20 nM, thus at markedly lower doses than the effective ones of Verapamil (50 mM).

Moreover, in accordance with the classification proposed by Polli et al. (supra), such compounds are classifiable as pure inhibitors of P-gp, since under the specific experimental conditions they behave as the reference compound GF120918 (Elacridal), i.e., they:

a) Are not transported by P-gp (overexpressed in tumor cell lines used as standard, in particular in CACO-2);

b) Do not activate the ATP-ase enzyme;

c) Inhibit the transport of P-gp specific substrates, like [³H]vinblastine and rhodamine.

On the contrary, compounds such as 3-({2-[2-(3-methoxyphenyl)ethyl]phenoxy}methyl)pyridine (EB18) and 1-(benzyloxy)-2-(2-phenylethyl)benzene (EB 30) act as substrates with different implications on P-gp activity (classifiable as Category IA and Category IIB, respectively).

Synthesis Process

The compounds of the invention have been produced as reported in the following synthetic schemes.

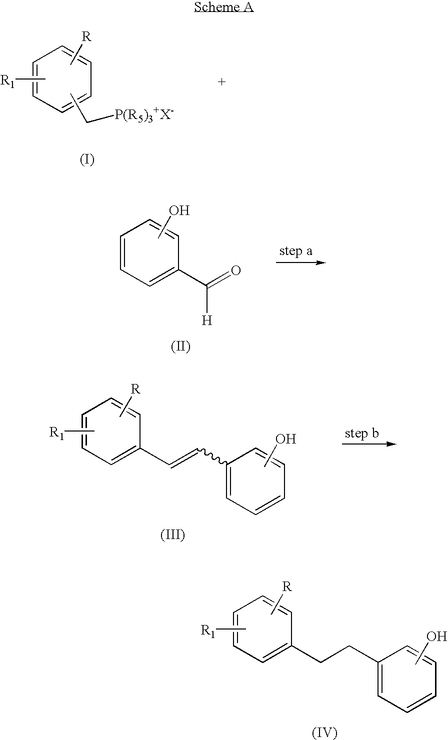

The reaction, illustrated in scheme A, of compound I with compound II, suitably substituted, has led to olefin derivative III that was subsequently reduced to derivative IV.

The meaning of R and R1 is the same as indicated above. They may independently be hydrogen atoms, a (C1-C4) alkyl group, a (C1-C3) alcoxy group, a halogen atom, a cyano group, a thiol group, a hydroxyl group, a (C1-C3) thioalkyl group. R5 is a phenyl or an alkyl; X represents a halogen atom.

Step a

The reaction of compound I with compound II has led to compound III. The reaction may be carried out in a wide range of temperatures; in fact, temperature is not critical to the reaction. In general, the reaction is conducted at a temperature range comprised between 0° C. to 200° C. The time required for the reaction may vary enormously and this depends on various factors, among which the reaction temperature, the nature of the solvents employed and the nature of the reagents. If the reaction is conducted under the conditions listed above, the reaction time may be comprised in the range of from 30 min to 24 h, e.g. 1, 3, 5, 10, 15 or 20 h. The reaction is preferably conducted in the presence of solvent. There are no particular restrictions on the nature of the solvent that may be used. Solvents useful in this reaction comprise nitriles, like acetonitrile; aromatic hydrocarbons, like toluene, benzene or xylene; amides, like dimethylacetamide, ethers, like tetrahydrofuran, dioxane or ethyl ether. The reaction occurs via the formation of an ilyde from derivative I; this compound (Ia) (wherein R₅ is a phenyl, an alkyl, and X is a halogen atom) is formed in situ in the presence of a base, e.g. an amine like 1,8-diazabicyclo[5,4,0]undec-7-ene or 1,5-diazabicyclo[4,3,0]non-5-ene; an alkaline hydroxide, like sodium hydroxide or potassium hydroxide; an amide, like sodium amide or potassium amide; an alkoxyde, like sodium methoxide, sodium ethoxide or potassium t-butoxide.

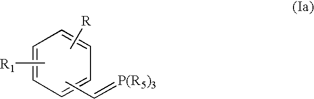

The temperature at which the reaction occurs may be comprised in the range of from 0° C. to 100° C., e.g. at 10° or at room temperature or at 40°, 60°, 75° C. In addition, the time required for the reaction varies enormously according to the nature of the solvent and of the reagents employed and to the reaction temperature. If the reaction is conducted under the conditions described above, the reaction time is comprised between 10 min and 24 h, e.g. 1, 3, 5, 10, 15 or 20 h.

At the end of the reaction, the desired compound of formula III may be recovered from the reaction mixture by means of conventional methods. E.g., a recovery procedure is represented by filtration of insoluble material (if present) and subsequent solvent evaporation under reduced pressure to give the desired product. An alternative is solvent evaporation under reduced pressure, and the residue is recollected with water and extracted with a water-immiscible organic solvent (e.g., ethyl acetate, dichloromethane). The reunited organic phases are dried with a dehydrating agent, like sodium sulphate, and finally the solvent is evaporated. The residue, if necessary, may be further purified by means of traditional methods such as crystallization or various chromatography techniques.

Step b

The Carbon-Carbon double bond of compound III may be reduced via catalytic hydrogenation (conducted at a pressure ranging from the atmospheric one to 10 atm). The reaction is preferably conducted in the presence of a solvent. There are no restrictions on the nature of the solvent to be used. Examples of solvents that may be used are alcohols, like methanol, ethanol or isopropanol; ethers, like ethyl ether, tetrahydrofuran or dioxane, aromatic hydrocarbons, like toluene, benzene or xylene; aliphatic hydrocarbons, like hexane or cyclohexane; esters, like ethyl acetate, and fatty acids, like acetic acid. A mixture of one or more of the above-listed solvents may also be used. The reaction is conducted in the presence of a catalyst, preferably palladium on coal or Raney nickel, platinum oxide, rhodium on alumina, triphenylphosphine-rhodium or palladium on barium sulphate. The reaction may occur within a wide range of temperatures, and temperature is not critical to the ends of the reaction. In general, it is more convenient to perform the reaction under temperature conditions ranging from 20 to 80° C., e.g. at room temperature or at 40°, 60° C. The time required for the reaction depends on the nature of the reagents and on the solvent used. Generally, under the above-described conditions, a time ranging from 30 min to 48 h, e.g. 1, 3, 5, 10, 15, 20 or 30 h, is sufficient.

At the end of the reaction, the desired compound of formula IV may be recovered from the reaction mixture by means of traditional methods. E.g., a recovery procedure is represented by filtration of insoluble material (if present) and subsequent solvent evaporation under reduced pressure to give the desired product. An alternative is solvent evaporation under reduced pressure, the residue being recollected with water and extracted with a water-immiscible organic solvent (e.g., ethyl acetate, dichloromethane). The reunited organic phases are dried with a dehydrating agent, like sodium sulphate, and lastly the solvent is evaporated. The residue, if necessary, may be further purified by means of conventional methods, such as crystallization or various chromatography techniques.

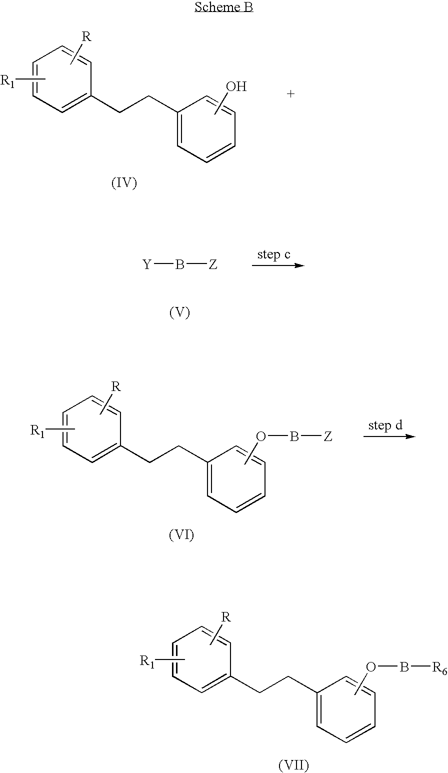

In the formulas describing this scheme, R, R1 are defined as already described above for scheme A. Group B represents an alkyl group with Carbon atoms ranging from 1 to 4; Y represents a halogen atom, Z may be a cyclic 5- or 6-membered group, like an aryl group, unsubstituted or substituted with a C1, C2, C3 alkyl group, or with a C1, C2, or C3 alkoxy group, or a 5- or 6-membered aromatic or aliphatic heterocyclic group, containing a nitrogen, oxygen or sulphur atom, even substituted, e.g. N-alkyl substituted or N-alkyl-aryl substituted, N-alkyl-halogenoaryl substituted, a halogen or a 3-piperidine protected on the amine nitrogen. R6 may have the same meaning of R2 in the general formula, in particular it may represent an aryl group, a 5- to 6-membered heteroaromatic group obtained from Z through the reactions indicated at step d.

R6 may also be an —NR3R4-type group, where R3 and R4 may independently be a hydrogen atom, (C1, C2, C3, C4, C5) alkyl groups, even substituted with 5- or 6-membered aromatic or heteroaromatic systems, or (C1, C2, C3, C4, C5) alkyl chains bound to the nitrogen to build a 5- or 6-membered aliphatic heterocycle, condensed or bound to a further aromatic or heteroaromatic group, it also 5- or 6-membered.

Compound IV was submitted to alkylation with the suitable alkylating agent V in the presence of a base.

Step c

A mole of compound IV was approximately reacted with the base (1 to 3 moles) and with the alkylating agent V (1-3 moles). The reaction is conventionally conducted in solvents like dimethylformamide, dimethylacetamide, dimethyl sulfoxide, acetonitrile, acetone, methyl ethyl ketone, and the like. Useful bases are sodium hydride, potassium t-butoxide, potassium carbonate, sodium carbonate and the like. Examples of alkylating agents may be alkyl halides (chlorides, bromides, iodides). Reaction conditions may vary and depend on the combination of the alkylating agent and the base used. The reaction is conducted at temperatures ranging from 0 to 100° C., e.g. 5°, 10°, 20°, 40°, 60°, 80° C., and depends on the nature of the reagents used and on the nature of the solvent and of the base used. Reaction times are influenced by the type of reagents and solvent used and may range from 1 to 48 h.

At the end of the reaction the desired compound of formula VI may be recovered from the reaction mixture by means of conventional methods. E.g., a recovery process is represented by filtration of insoluble material (if present) and subsequent solvent evaporation under reduced pressure to afford the desired product. An alternative is solvent evaporation under reduced pressure, the residue being recollected with water and extracted with a water-immiscible organic solvent (e.g. ethyl acetate, dichloromethane). The reunited organic phases are dried with a dehydrating agent, like sodium sulphate, and lastly the solvent is evaporated. The residue, if necessary, may be further purified by means of conventional methods such as crystallization or various chromatography techniques.

Step d

The reaction described in this step may be an (a) amine group alkylation or acylation reaction, (b) nucleophilic substitution reaction with an amine of an alkyl halide, (c) elimination of a protecting group on the amine nitrogen and/or subsequent alkylation or acylation reaction described at (a) of step d. In fact, N-unsubstituted piperidine compounds are not covered by this patent application.

Reaction (a): the amine group alkylation or acylation reaction is conducted by using alkylating or acylating agents in the presence of a base. This reaction is essentially analogous to that described at step c and may be carried out by using the same reactants and the same reaction conditions.

Reaction (b): In this reaction, the alkyl halide VI is submitted to a nucleophilic substitution reaction with suitable amine derivatives. The experimental procedure followed for this reaction is analogous to that described in reaction (a) of step C and it may be carried out by using the same reagents and the same reaction conditions.

Reaction (c): In this reaction, it is eliminated the protecting group on the amine nitrogen. The nature of the deprotection reactions depends on the features of the protecting group employed. When the protecting group is a t-butoxycarbonyl, elimination of this group occurs by reacting the compound having the protecting group with an inorganic acid, like hydrochloric acid, nitric acid, or sulphuric acid; an organic acid, like acetic acid, trifluoroacetic acid, methanesulfonic acid, or p-toluensulfonic acid; a Lewis acid, like boron trifluoride. The reaction is conducted in the presence of a solvent; there are no particular restrictions limiting the solvent selection. Examples of solvents used are hydrocarbons, like benzene or hexane; halogenated hydrocarbons, like methylene chloride, or chloroform; esters, like ethyl acetate; ketons, like acetone or methyl ethyl ketone; alcohols, like methanol or ethanol; ethers, like ethyl ether, tetrahydrofuran or dioxane; a mixture of the above-listed solvents thereamong or with water. The reaction may occur within a wide range of temperatures, comprised between −10° C. and 100° C. Also the reaction time may vary, and it depends on the nature of the solvent and of the reagents employed, and on the reaction temperatures. If the reaction is conducted under the above-listed conditions, a reaction time comprised between 30 min and 48 h is considered sufficient. At the end of the reaction, the desired compound is recovered from the reaction mixture by means of conventional methods. E.g., the mixture may be neutralized, the inorganic salts filtered and the filtrate diluted with water and water-immiscible organic solvents, like ethyl acetate. The organic extracts are then washed with water, dried with dehydrating agents like magnesium sulphate or sodium sulphate, and evaporated. The obtained compound could subsequently be purified by the usual purification techniques, such as crystallization, precipitation, and various chromatography techniques.

When the protecting group of the nitrogen is an alcoxycarbonyl residue, this can be eliminated by submitting the protected compound to hydrolysis using a base, preferably alkaline hydroxides like lithium hydroxide, sodium hydroxide or potassium hydroxide; or to bases such as carbonates, like sodium carbonate, or potassium carbonate. The reaction is conducted in the presence of a solvent. Examples of solvents employed are alcohols, like methanol or ethanol, ethers, like tetrahydrofuran or dioxane; water; a mixture of one or more of the abovementioned organic solvents thereamong or with water. The reaction may occur at different temperatures; generally, the reaction is conveniently conducted at temperatures ranging from 0° C. to 100° C. The reaction time depends on various factors, such as the temperature and the nature of the solvents and of the reagents used; it may vary within a range of from 30 min to 48 h. At the end of the reaction, the product may be recovered from the reaction mixture by using the methods described hereto.

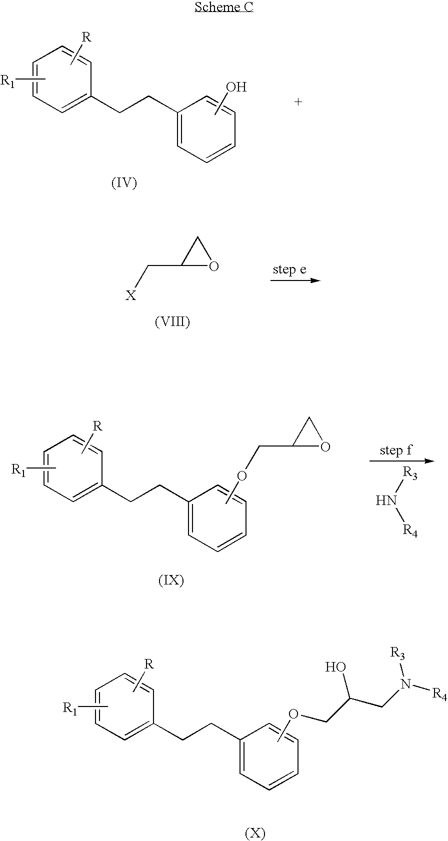

The substituents R, R1, X, R3 and R4 present in the formulas indicated in scheme C have the same meaning defined in the preceding schemes.

Step e

In step e, a compound of formula IX was obtained by reaction of the compound IV with a compound of structure VIII. This reaction is essentially analogous to that described in scheme B by step C, and may be carried out by using the same reagents and the same reaction conditions.

Step f

The compound of formula IX is reacted with an amine derivative of formula NHR₃R₄ indicated in scheme C.

The reaction is preferably conducted in the presence of solvent. There are no particular restrictions concerning the type of solvent used, with the exception of the insolubility of the reagents involved in the reaction. Examples of solvents employed are hydrocarbons, like hexane, benzene or toluene; halogenated hydrocarbons, like methylene chloride, chloroform, 1,2-dichloroethane; ethers, like ethyl ether, tetrahydrofuran, dioxane; ketons, like acetone, ethylmethylketone; nitrites, like acetonitrile; amides, like dimethylacetamide, dimethylformamide, N-methyl-2-pyrrolidone; sulfoxides, like dimethylsulfoxide; and water. There may be used only one of the above-listed solvents or a mixture thereof. The reaction occurs in a wide range of temperatures comprised between −10° C. and 100° C. The reaction time may vary and depends on different factors, among which the reaction temperature, the nature of the reagents and of the solvents used. The reaction time in the above-listed conditions may vary in a range comprised between 30 min and 48 h.

At the end of the reaction, the desired compound may be recovered from the reaction mixture by means of conventional methods, among which solvent evaporation, or product extraction by means of water-immiscible organic solvents. The extracts are then dried with dehydrating agents such as magnesium sulphate or sodium sulphate and evaporated. If necessary, the product may be purified by means of conventional methods such as crystallization, precipitation, or chromatography techniques.

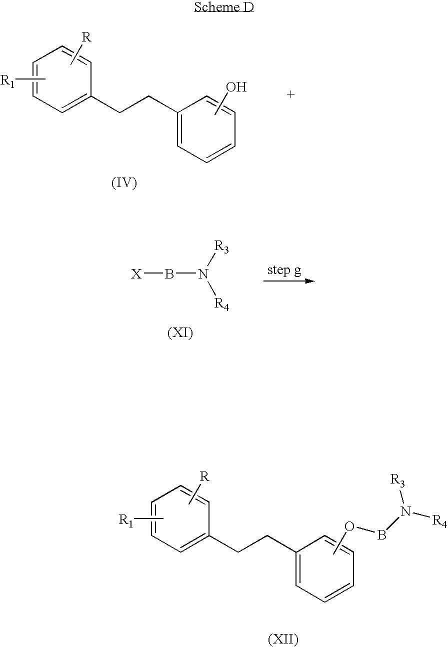

The substituents R, R1, R3 and R4 present in the formulas indicated in scheme D have the same meaning indicated in the preceding schemes. Group X may be a halogen, an alkansulfonyloxy group or an arylsulfonyloxy group; B may be an alkyl chain comprised between 1 and 4 carbon atoms or a single or repeating —CH₂—CH(OH)—CH₂— chain.

Step g

The compound reported in scheme D is prepared by reaction of compound IV with derivative XI. Group X represents an atom or a group able to behave as good leaving group in nucleophilic substitution reactions. Among these groups, X may be a halogen atom, an alkansulfonyloxy group or an arylsulfonyloxy group; the reaction is generally conducted in the presence of inert solvents and in the presence of a base.

The base used may be of various type; the more commonly used ones are alkali metal carbonates, like sodium carbonate, potassium carbonate; alkali metal hydrogen carbonates, like sodium hydrogen carbonate or potassium hydrogen carbonate; alkali metal fluorides, like sodium fluoride and potassium fluoride; hydrides, like sodium hydride, potassium hydride or lithium hydride; alkoxides, like sodium methoxide, sodium ethoxide, potassium t-butoxide, lithium methoxide; organic amines, like pyridine, pycoline, triethylamine, N-methylmorpholine, or 4-dimethylaminopyridine. The reaction is preferably conducted in the presence of a solvent. There are no particular restrictions concerning the type of solvent that may be used in this reaction, with the exception of the solubility of the reagents. Examples of solvents used in this reaction are represented by hydrocarbons, like hexane, toluene or benzene; preferably aliphatic halogenated hydrocarbons, like methylene chloride, chloroform, 1,2-dichloroethane; ethers, like diethyl ether, tetrahydrofuran, dioxane; ketons, like acetone, methyl ethyl ketone; nitriles, like acetonitrile; amides, like dimethylformamide, dimethylacetamide, and sulfoxides, like dimethylsulfoxide. There may be used only one of the above-listed solvents or a mixture thereof. The reaction occurs in a wide range of temperatures, comprised between 0° C. and 100° C. The reaction time may vary and depends on different factors, among which the reaction temperature, the nature of the reagents and of the solvents used. The reaction time under the above-listed conditions may vary in a range comprised between 30 min and 48 h.

When group X is represented by a hydroxyl group, the reaction in this step is preferably conducted in an inert solvent and in the presence of triphenylphosphine and azodicarboxyl acid esters, like dimethylazodicarboxylate or diethylazodicarboxylate. The solvents used may be aromatic hydrocarbons, halogenated hydrocarbons or an ether. The reaction occurs in a wide range of temperatures, comprised between −20° C. and 100° C. The reaction time may vary and depends on different factors, among which the reaction temperature, the nature of the reagents and of the solvents used. The reaction time under the above-listed conditions may vary in a range comprised between 30 min and 48 h.

At the end of the reaction, the desired compound may be recovered from the reaction mixture by means of conventional methods, among which solvent evaporation, or product extraction by means of water-immiscible organic solvents, following evaporation of the reaction solvent. The extracts are then dried with dehydrating agents such as magnesium sulphate or sodium sulphate and evaporated. If necessary, the product may be purified by means of conventional methods, such as crystallization, precipitation, or of chromatography techniques.

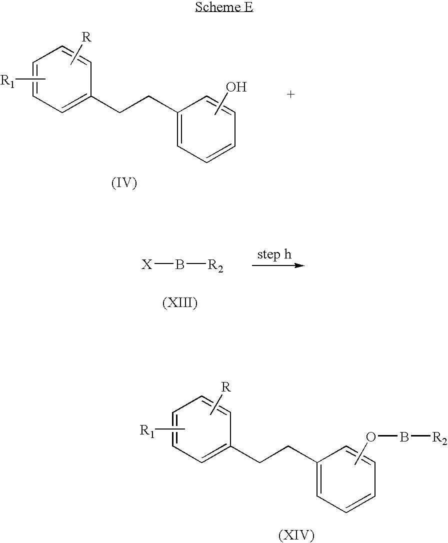

wherein R and R1 and R2 and B have the same meaning already indicated in the preceding schemes and in the general formula.

Step h

The compound described in scheme E is obtained by reaction of compound IV with a compound of formula XIII. This reaction is substantially analogous to that described in step c of scheme B and may be conducted by using the same reagents and the same reaction conditions.

Therapeutic Applications

The compounds of the invention able to inhibit P-gp activity find therapeutic application in two relevant and different fields: the first one in the oncological context, the second one in the context of the treatment of pathologies related to the central nervous system (CNS).

In particular, concerning the oncological context, the therapeutic usefulness of compounds able to inhibit P-gp is linked to the treatment of tumor pathologies that have become resistant to the action of chemotherapy agents. Drug resistance is certainly a therapeutic limitation conditioning the pharmacological strategy. Many studies have been carried out on this issue, and the research results, by now established under the biochemical, pharmacokinetic and pharmacodynamic standpoints, singled out in P-gp overexpression the cause of drug resistance. This protein, which is a membrane ATP-ase pump in charge of the cellular efflux both of hexogen compounds and cell metabolites, in some tumor pathologies, in particular like colon and mammary carcinoma, is overexpressed, thereby increasing the expulsion from the cell of the chemotherapy agent, whose effect will be increasingly less effective with the increase of the ATP-ase activity of the pump. Availability of pure inhibitors of P-gp allows to attain, above all in resistant tumor forms, improved pharmacological effectiveness of the chemotherapy agent to which the preceding treatment had induced drug resistance. Of course, for each P-gp inhibitor compound it is important to ascertain that there are no undesired side effects, like cytotoxicity, or the involvement of other ATP-ase systems present inside (SERCA) and onto (PMCA) the cell membrane in charge of Ca⁺⁺ intracellular homeostasis.

Concerning the application of P-gp inhibitors in the context of CNS-related pathologies, it has to be taken into account that the reaching of the biological target by drugs active in the CNS is regulated by the action of the blood-brain barrier (BBB). The active component of this barrier is composed of the P-glycoprotein that therefore limits the access, and reduces the presence, of molecules in the CNS. This entails, in the treatment of CNS pathologies, the need to administer high doses of drug so that part of it may reach the CNS, whereas the remainder carries out a saturating effect on the extrusion pump represented by the P-gp present at the BBB level. Accordingly, availability of pure and reversible inhibitors of the P-glycoprotein present at the BBB level allows a pharmacological treatment of CNS pathologies with much smaller therapeutic doses and the entailed reduction of undesired effects.

The invention is detailed hereinafter by means of examples, which however have a merely illustrative and non-limiting purpose.

### EXAMPLE 1

### Evaluation of Inhibition Activity of Compounds EB25, EB27 and EB12

The compounds under examination (EB25, EB27 and EB12) at a 20 μM final concentration in complete medium (DMEM, 10% FCS) were added for 120 min to 20000 MCF7/Adr (doxorubicin-resistant human breast carcinoma cells). At the end of the incubation, the incubation medium containing the compound under examination was removed, and fresh medium containing 50 μM doxorubicin was added for 24 h. The negative control consisted of plates containing an alike number of cells untreated with the compound. At the end of the 24 h, the medium was removed and the cells split and prepared according to standard procedure for flow cytometry analysis. The results, illustrated in FIGS. 1, 2 and 3, highlight that the 120-min pretreatment with compounds EB25 (FIG. 1), EB27 (FIG. 2) and EB12 (FIG. 3) and subsequent treatment with doxorubicin leads to an increase in the inletting of the latter in the cells. Such an increase is highlighted by the shifting of the doxorubicin curve, with respect to that of doxorubicin on a pretreated sample, and this effect, concentrations being equal, is more marked for EB25 and EB12. The same experiment was carried out with Verapamil (unreported data), reference compound, and the results highlighted that the effect of Verapamil pretreatment leads to in-cell doxorubicin increase of the same order of the compound EB27. Compound EB25 was about 10-fold, and compound EB12 was 5-fold more effective than Verapamil and EB27 in the P-glycoprotein inhibition effect in the studied cell line.

### EXAMPLE 2

### (3-METHOXY)BENZYLTRIPHENYLPHOSPHORANE CHLORIDE SYNTHESIS (compound I, scheme A, intermediate)

A solution of 3-methoxybenzylchloride (5 g, 32 mmoles) and PPh₃ (9.205 g, 33.93 mmol) in CH₃CN (20 mL) was kept under stirring for 12 h under reflux. Thereafter, the solvent was evaporated under reduced pressure, the obtained compound was recollected with the minimum amount of CHCl₃ and precipitated with ether.

The solid formed was filtered, giving the desired compound (98.5%).

¹H NMR(CDCl₃): δ 3.53 (s, 3H, OCH₃); 5.44 (d, 2H, J=−16 Hz, CH₂); 6.60-6.77 (m, 3H, Ar); 7.02 (t, 1H, J=7.6 Hz,); 7.11-7.21 (m, 15H, PPh₃) ppm.

MS (m/z): M⁺ —Cl 383 (16.1%)

Elemental Analysis:

| C₂H₂₄OPCl | C | H |
|---|---|---|
| Calculated % | 74.19 | 6.23 |
| Found % | 74.28 | 6.40 |

### EXAMPLE 3

### 2-[(E/Z)-2-(3-METHOXYPHENYL)VINYL]PHENOL SYNTHESIS (compound III, scheme A, intermediate)

1.8-diazabicyclo(5.4.0)-undec-7-ene (DBU) (2.30 ml) was added to a solution of (3-methoxy)benzyltriphenylphosphorane chloride (6.05 g, 14.8 mmol) and salicylaldehyde (1.81 g, 14.8 mmol) in CH₃CN (22.7 ml). The reaction mixture was refluxed for 12 h, under stirring; thereafter the solvent was evaporated and the crude product, recollected with CHCl₃, was submitted to washes with H₂O and an 1N aqueous HCl solution.

The organic phase, dried, filtered and evaporated under reduced pressure, afforded a yellow oil that was not submitted to any further purification.

Yield: 89%

¹H NMR(CDCl₃): δ 3.84 (s, 3H, OCH₃); 6.55-6.93 (m, 4H, Ar); 6.96-7.23 (m, 6H, Ar, CH═CH) ppm.

### EXAMPLE 4

### 2-[2-(3-METHOXYPHENYL)ETHYL]PHENOL SYNTHESIS (compound IV, scheme A intermediate)

A solution of stilbene derivative (6.61 g, 28.9 mmol) in abs. EtOH. (146.44 ml), was submitted to hydrogenation using 10% Pd/C (444 mg) as catalyst for 24 h. Thereafter, the catalyst was filtered on celite and the solvent was evaporated under reduced pressure, obtaining an oil that was purified by column chromatography using CHCl₃ (40%) as eluent.

¹H NMR(CDCl₃): δ 2.91 (s, 4H, CH₂CH₂); 3.78 (s, 3H, OCH₃); 6.71-6.94 (m, 4H, Ar); 7.04-7.28 (m, 4H, Ar) ppm.

### EXAMPLE 5

### 3-({2-[2-(3-METHOXYPHENYL)ETHYL]PHENOXY}METHYL)PYRIDINE SYNTHESIS (EB18)

A solution of KOH (174.4 mg, 3.11 mmol) and DMSO was heated to 80°, to complete dissolution of KOH. The solution was cooled to room temperature and phenol (200 mg, 0.87 mmol) dissolved in a minimum amount of DMSO was added thereto. The mixture was left under stirring at room temperature. After 10 min, suitable pycoline (0.87 mmol), dissolved in a minimum amount of DMSO, was added to the solution of potassium phenate. The solution was stirred for 12 h, under reflux. Thereafter, the solution was recollected with AcOEt and submitted to washes with H₂O (3), NaOH (2) and NaCl (2). The organic phase was dried and evaporated.

The product was purified by transformation to oxalate.

Yield: 36%

¹H NMR(CDCl₃): δ 2.80-2.98 (m, 4H, CH₂); 3.73 (s, 3H, OCH₃), 5.08 (s, 2H, OCH₂); 6.66-6.96 (m, 4H, Ar); 7.01-7.26 (m, 5H, Ar+Py); 7.30-7.37 (m, 1H, Py); 7.76-7.81 (m, 1H, Py); 8.59 (d, J=4.0 Hz, 1H, Py); ppm.

MS (m/z): M⁺ 319 (100%); 227 (12%); 212 (100%).

mp: 132-134° C.

Elemental Analysis:

| C₂₁H₂₁O₂N•H₂C₂O₄ | N | C | H |
|---|---|---|---|
| Calculated % | 3.42 | 67.48 | 5.42 |
| Found % | 3.15 | 67.12 | 5.03 |

### EXAMPLE 6

### 2-({2-[2-(3-METHOXYPHENYL)ETHYL]PHENOXY}METHYL)PYRIDINE (EB12) SYNTHESIS

A solution of KOH (174.4 mg, 3.11 mmol) and DMSO was heated to 80°, to complete dissolution of KOH. The solution was cooled to room temperature and phenol (200 mg, 0.87 mmol) dissolved in a minimum amount of DMSO was added thereto. The mixture was left under stirring at room temperature. After 10 min, 3-chloropycoline (110 mg, 0.87 mmol), dissolved in a minimum amount of DMSO, was added to the solution of potassium phenate. The solution was stirred for 12 h under reflux. Thereafter, the solution was recollected with AcOEt and submitted to washes with H₂O (3), NaOH (2) and NaCl (2). The organic phase was dried and evaporated.

The product was purified by transformation to oxalate and subsequent crystallization from iPrOH.

Yield: 22%

¹H NMR(CDCl₃):δ 2.90-3.01 (m, 4H, CH₂—CH₂); 3.75 (s, 3H, OCH₃); 5.29 (s, 2H, OCH₂); 6.72-6.96 (m, 4H, Ar); 7.14-7.39 (m, 5H, Ar+Py); 7.63 (d, 1H, J=8.05 Hz, Py); 7.86 (t, 1H, J=7.9 Hz, Py); 8.66 (d, 1H, J=4.9 Hz, Py) ppm.

MS (m/z): M⁺ 319 (6%); 228 (3%)

mp: 119-121° C.

Elemental Analysis:

| C₂₁H₂₁NO₂•H₂C₂O₄ | N | C | H |
|---|---|---|---|
| Calculated % | 1.93 | 49.72 | 4.42 |
| Found % | 1.78 | 50.17 | 4.76 |

### EXAMPLE 7

### 1-(3-BROMOPROPOXY)-2-(2-PHENYLETHYL)BENZENE DERIVATIVE SYNTHESIS (Scheme b, step c, compound VI, intermediate)

A solution of t-BuOH (11.95 ml) and KOH (502.09 mg; 8.95 mmol) was heated to 80° C. to complete dissolution of KOH. The solution was cooled to room temperature, and phenol (1 g; 4.38 mmol) previously dissolved in a minimum amount of t-BuOH, was added dropwise. In another flask, a solution of 1,3-dibromopropane (6.57 mmol) in t-BuOH (minimum amount) was prepared and kept under stirring at room temperature. By a dropping funnel, the solution of potassium phenate was added to the solution of 1,3-dibromopropane in t-BuOH, in an ice bath and under stirring. The mixture was kept under stirring at room temperature for 24 h. Thereafter, t-BuOH was evaporated under reduced pressure and the crude product, recollected with Et₂O, was submitted to washes with H₂O. The organic phase, dried and filtered, was evaporated under reduced pressure.

Yield: 88%

¹H NMR(CDCl₃): δ 2.35 (quint., 2H, J=6.4 Hz, CH₂); 2.87-2.93 (m, 4H, CH₂); 3.63 (t, 2H, J=6.4 Hz, CH₂Br); 3.79 (s, 3H, OCH₃); 4.11 (t, 2H, J=5.8 Hz, OCH₂); 6.73-6.92 (m, 4H, Ar); 7.04-7.26 (m, 4H, Ar) ppm.

### EXAMPLE 8

### N-(2-ETHYLPYRIDINE)-3-{2-[2-(3-METHOXYPHENYL)ETHYL]PHENOXY}PROPAN-1-AMINE (EB17) SYNTHESIS

Aryloxypropyl derivative (200 mg; 0.57 mmol), dissolved in a minimum amount of CH₃CN was added to a solution of K₂CO₃ (142.54 mg; 1.03 mmol) in CH₃CN (15 ml), kept under stirring at 80° C. Lastly, 2-pyridinylethanamine (85 mg; 0.68 mmol), previously dissolved in a minimum amount of CH₃CN, was added to the solution.

The mixture was kept under stirring at room temperature for 12 h. Thereafter, K₂CO₃ was filtered on a septum and the solid was washed with CH₂Cl₂. The solvent was evaporated under reduced pressure.

The product was purified by transformation to the suitable oxalate.

Yield: 35%

¹H NMR (DMSO): δ 2.10-2.38 (m, 2H, CH₂); 2.80 (s, 4H, CH₂₋CH₂), 3.10-3.18 (m, 4H, CH₂₋CH₂); 3.32-3.39 (m, 2H, CH₂); 3.68 (s, 3H, OCH₃); 4.02-4.13 (m, 2H, CH₂); 6.71-6.97 (m, 4H, Ar); 7.16-7.34 (m, 6H, Ar, Py); 7.73-7.80 (m, 1H, Py); 8.47-8.50 (m, 1H, Py) ppm.

MS (m/z): M⁺ 390 (25%); 298 (18%).

mp: 154-156° C.

Elemental Analysis:

| C₂₅H₃₀O₂N₂•2H₂C₂O₄ | N | C | H |
|---|---|---|---|
| Calculated % | 4.91 | 61.05 | 5.90 |
| Found % | 5.07 | 60.81 | 5.58 |

### EXAMPLE 9

### 1-(3-CHLOROPHENYL)-4-(3-{2-[2-(3-METHOXYPHENYL)ETHYL]PHENOXY}PROPYL)-PIPERAZINE (MC 71) SYNTHESIS

A solution of KOH (0.44 g, 3.19 mmol) and DMSO was heated to 80°, to complete dissolution of KOH. The solution was cooled to room temperature and phenol (0.5 g, 2.19 mmol) dissolved in a minimum amount of DMSO was added thereto. The mixture was left under stirring at room temperature. After 10 min, 1-(3-chlorophenyl)piperazine (2.19 mmol), dissolved in a minimum amount of DMSO, was added to the solution of potassium phenate. The solution was kept under stirring and under reflux for 20 h. Thereafter, the solution was recollected with AcOEt and submitted to washes with H₂O (3), NaOH (2) and NaCl (2). The organic phase was dried and evaporated.

The product was purified by transformation to hydrochloride and subsequent crystallization from iPrOH.

Yield: 90%

¹H NMR(CDCl₃):δ 2.02-2.12 (m, 2H, CH₂); 2.59-2.70 (m, 6H, CH₂CH₂ pip.+ CH₂N); 2.85-2.93 (m, 4H, CH₂CH₂); 3.20 (t, 4H, J=4.95 Hz, CH₂CH₂ pip.); 3.78 (s, 3H, OCH₃); 4.05 (t, 2H, J=6.04 Hz, OCH₂); 6.73-6.91 (m, 8H, Ar); 7.11-7.25 (m, 4H, Ar) ppm.

MS (m/z): M⁺ 464 (40); 209 (100)

mp: 135-140° C.

Elemental Analysis:

| C₂₈H₃₄N₂O₂Cl₂ | N | C | H |
|---|---|---|---|
| Calculated % | 5.59 | 67.07 | 6.79 |
| Found % | 5.49 | 67.18 | 7.17 |

### EXAMPLE 10

### 4-METHYL-1-(3-{2-[2-(3-METHOXYPHENYL)ETHYL]PHENOXY}PROPYL)-PIPERAZINE (MC 72) SYNTHESIS

The product was synthesized as reported in example 9 and purified by transformation to hydrochloride and subsequent crystallization from EtOH.

Yield: 47.4%

¹H NMR(DMSO): δ 2.10-2.32 (m, 2H, CH₂); 2.51-2.95 (m, 7H, CH₂CH₂+ CH₃), 3.10-3.67 (m, 10H, CH₂CH₂ pip.+ CH₂CH₂ pip.+ CH₂N); 3.71 (s, 3H, OCH₃); 4.06 (t, 2H, J=5.9 Hz, OCH₂); 6.70-7.05 (m, 5H, Ar); 7.09-7.35 (m, 3H, Ar) ppm.

MS (m/z): M⁺ 368 (22); 149 (100)

mp: 194-198° C.

Elemental Analysis:

| C₂₃H₃₈N₂O₂Cl | N | C | H |
|---|---|---|---|
| Calculated % | 5.87 | 57.86 | 7.97 |
| Found % | 5.53 | 57.47 | 7.68 |

### EXAMPLE 11

### N-(3-TRIFLUOROMETHYL)PHENYL-1-(3-{2-[2-(3-METHOXYPHENYL)ETHYL]PHENOXY}PROPYL)-PIPERAZINE (EC68) SYNTHESIS

The aryloxypropyl derivative (200 mg; 0.57 mmol), dissolved in a minimum amount of CH₃CN was added to a solution of K₂CO₃ (94.4 mg; 0.68 mmol) in CH₃CN (15 ml), kept under stirring at 80° C. Lastly, the piperazine derivative (0.57 mmol) previously dissolved in a minimum amount of CH₃CN was added to the solution. The mixture was kept under stirring at 80° C., for 4 h, under reflux. Thereafter, the K₂CO₃ was filtered on a septum and the solid was washed with CH₂Cl₂. The solvent was evaporated under reduced pressure.

The product was purified by transformation to hydrochloride and subsequent crystallization from iPrOH.

Yield: 33.9%

¹H NMR(CDCl₃): δ 2.40-2.51 (m, 2H, CH₂); 2.80-2.90 (m, 4H, CH₂CH₂); 3.15-3.31 (m, 4H, CH₂CH₂ pip.); 3.35-3.50 (m, 2H, CH₂N); 3.51-3.70 (m, 4H, CH₂+ CH₂ pip.); 3.73 (s, 3H, OCH₃); 4.01-4.12 (m, 2H, OCH₂); 6.70-6.87 (m, 4H, Ar); 6.93 (t, 1H, J=7.3 Hz, Ar); 7.12-7.24 (m, 5H, Ar); 7.35-7.48 (m, 2H, Ar) ppm.

MS (m/z): M⁺ 498 (18); 188 (100)

mp: 128-135° C.

Elemental Analysis:

| C₂₉H₃₆N₂O₃F₃Cl | N | C | H |
|---|---|---|---|
| Calculated % | 5.07 | 62.99 | 6.52 |
| Found % | 6.54 | 58.20 | 5.62 |

### EXAMPLE 12

### N-(4-FLUOROPHENYL)-1-(3-{2-[2-(3-METHOXYPHENYL)ETHYL]PHENOXY}PROPYL)-PIPERAZINE (EC67) SYNTHESIS

The product was synthesized as described in example 11 and purified by transformation to hydrochloride and subsequent crystallization from iPrOH.

Yield: 68.7%

¹H NMR(CDCl₃): δ 2.32-2.50 (m, 2H, CH₂); 2.81-2.91 (m, 4H, CH₂CH₂); 3.23-3.70 (m, 2H, CH₂N); 3.50-3.70 (m, 4H, CH₂CH₂ pip.); 3.75 (s, 3H, OCH₃); 3.96-4.20 (m, 4H, CH₂CH₂ pip.); 4.61 (t, 2H, J=12.1 Hz, OCH₂); 6.68-6.97 (m, 5H, Ar); 7.15-7.24 (m, 5H, Ar); 7.77-7.84 (m, 2H, Ar) ppm.

MS (m/z): M⁺ 448 (89); 193 (100)

mp: 120-127°CA

Elemental Analysis:

| C₃₁H₄₃N₂O₃FCl₂ | N | C | H |
|---|---|---|---|
| Calculated % | 4.82 | 63.81 | 7.38 |
| Found % | 4.99 | 61.79 | 6.94 |

### EXAMPLE 13

### N-(2-METHOXYPHENYL)-1-(3-{2-[2-(3-METHOXYPHENYL)ETHYL]PHENOXY}PROPYL)-PIPERAZINE (EC65) SYNTHESIS

The product was synthesized as described in example 11 and purified by transformation to hydrochloride and subsequent crystallization from iPrOH.

Yield: 61.3%

¹H NMR(CDCl₃): δ 2.39-2.49 (m, 2H, CH₂); 2.82-2.94 (m, 4H, CH₂CH₂); 3.21-3.35 (m, 2H, CH₂N); 3.49-3.61 (m, 4H, CH₂CH₂ pip.); 3.76 (s, 3H, OCH₃); 4.01-4.10 (m, 5H, OCH₂+ OCH₃); 4.29-4.44 (m, 2H, CH₂ pip.); 5.04 (t, 2H, J=11,5 Hz, CH₂ pip.); 6.67-6.83 (m, 4H, Ar); 6.92 (t, 1H, J=7.05 Hz, Ar); 7.02-7.21 (m, 5H, Ar); 7.46 (t, 1H, J=8.5 Hz, Ar); 8.19 (d, 1H, J=7.9 Hz, Ar) ppm.

MS (m/z): M⁺ 460 (66); 205 (100)

mp: 114-125° C.

Elemental Analysis:

| C₂₉H₄₀N₂O₄Cl | N | C | H |
|---|---|---|---|
| Calculated % | 5.08 | 63.2 | 7.23 |
| Found % | 5.50 | 62.40 | 6.96 |

### EXAMPLE 14

### GENERAL PROCEDURE FOR N-ARIL-1-(2-{2-[2-(3-METHOXYPHENYL)ETHYL]PHENOXY}ETHYL)-PIPERAZINE DERIVATIVES SYNTHESIS (COMPOUNDS XIV, SCHEME E, STEP h)

A solution of KOH (88.2 mg; 1.6 mmol) in DMSO (10 ml) was heated to 80° C. to complete dissolution of KOH. The solution was cooled to room temperature and the phenol derivative IV (102.6 mg; 0.45 mmol), previously dissolved in a minimum amount of DMSO, was added dropwise thereto. The reaction mixture was kept under stirring at room temperature for 15 min. The suitable ethyl piperazine chain was added to the solution of potassium phenate, and the mixture, kept under stirring, was heated to 40° C. for 1 h. Thereafter, the mixture was recollected with AcOEt and submitted to washes with H₂O (3), NaOH (2) and NaCl (2). The organic phase, dried and filtered, was evaporated under reduced pressure

### N-(3-CHLORO)PHENYL-1-(2-{2-[2-(3-METHOXYPHENYL)ETHYL]PHENOXY}ETHYL)-PIPERAZINE (EC43)

The product was synthesized as described in example 14 and purified by transformation to hydrochloride and subsequent crystallization from iPrOH/iPr₂O.

Yield: 70%

¹H NMR(CDCl₃): δ 2.74-2.82 (m, 4H, CH₂CH₂ pip.); 2.85-2.93 (m, 6H, CH₂CH₂+ CH₂N); 3.17-3.25 (m, 4H, CH₂CH₂ pip.); 3.78 (s, 3H, OCH₃); 4.16 (t, 2H, J=5.6 Hz, OCH₂); 6.75-6.90 (m, 7H, Ar); 7.16-7.25 (m, 5H, Ar) ppm.

MS (m/z): M⁺ 450 (5); 209 (100)

mp: 80-90° C.

Elemental Analysis:

| C₂₇H₃₂N₂O₂Cl₂ | N | C | H |
|---|---|---|---|
| Calculated % | 5.75 | 66.53 | 6.57 |
| Found % | 5.85 | 64.89 | 6.43 |

### N-(3-TRIFLUOROMETHYL)PHENYL-1-(2-{2-[2-(3-METHOXYPHENYL)ETHYL]PHENOXY}ETHYL)-PIPERAZINE (EC57)

The crude product was synthesized as described in example 14 and purified by column chromatography, using the hexane/acetate mixture in a 1:1 ratio as eluent, and by subsequent transformation to hydrochloride.

Yield: 86.2%

¹H NMR(CDCl₃): δ 2.78 (t, 4H, J=4.75 Hz, CH₂CH₂ pip.); 2.85-2.94 (m, 6H, CH₂CH₂+ CH₂N); 3.24 (t, 4H, J=4.75 Hz, CH₂CH₂ pip.); 3.78 (s, 3H, OCH₃); 4.17 (t, 2H, J=5.4 Hz, OCH₂); 6.73-6.94 (m, 5H, Ar); 7.03-7.24 (m, 6H, Ar); 7.34 (t, 1H, J=7.85 Hz, Ar) ppm.

MS (m/z): M⁺ 484 (5); 243 (100)

mp: 55-60° C.

Elemental Analysis:

| C₂₈H₃₆N₂O₃F₃Cl₂ | N | C | H |
|---|---|---|---|
| Calculated % | 4.79 | 57.53 | 6.16 |
| Found % | 4.71 | 56.72 | 5.34 |

### N-(4-FLUORO)PHENYL-1-(2-{2-[2-(3-METHOXYPHENYL)ETHYL]PHENOXY}ETHYL)-PIPERAZINE (EC66)

The crude product was synthesized as described in example 14 and purified by column chromatography using the hexane/acetate mixture in a 1:1 ratio as eluent, and by subsequent transformation to hydrochloride.

Yield: 70%

¹H NMR(CDCl₃): δ 2.75-2.81 (m, 4H, CH₂CH₂ pip.); 2.88-2.94 (m, 6H, CH₂N+CH₂CH₂); 3.09-3.14 (m, 4H, CH₂CH₂ pip.); 3.77 (s, 3H, OCH₃); 4.16 (t, 2H, J=5.6 Hz, OCH₂) 6.73-7.00 (m, 9H, Ar); 7.12-7.22 (m, 3H, Ar) ppm.

MS (m/z): M⁺ 434 (6); 193 (100)

mp: 72-80° C.

Elemental Analysis:

| C₂₇H₃₅N₂O₃FCl₂ | N | C | H |
|---|---|---|---|
| Calculated % | 5.33 | 61.71 | 6.67 |
| Found % | 5.27 | 61.50 | 6.27 |

### N-(2-METHOXY)PHENYL-1-(2-{2-[2-(3-METHOXYPHENYL)ETHYL]PHENOXY}ETHYL)-PIPERAZINE (EC52)

The product was synthesized as described in example 14 and purified by transformation to hydrochloride and subsequent crystallization from iPrOH.

Yield: 77.6%

¹H NMR(CDCl₃): δ 2.69-2.87 (m, 4H, CH₂CH₂ pip.); 2.88-3.00 (m, 4H, CH₂CH₂); 3.05-3.19 (m, 4H, CH₂CH₂ pip.); 3.68 (t, 2H, J=5.4 Hz, CH₂N); 3.77 (s, 3H, OCH₃); 3.86 (s, 3H, OCH₃); 4.17 (t, 2H, J=5.8 Hz, CH₂O); 6.73-6.98 (m, 7H, Ar); 7.05-7.21 (m, 5H, Ar) ppm.

MS (m/z): M⁺ 446 (2); 123 (45)

mp: 93-98° C.

Elemental Analysis:

| C₂₈H₄₀N₂O₅Cl₂ | N | C | H |
|---|---|---|---|
| Calculated % | 5.04 | 60.50 | 7.20 |
| Found % | 4.56 | 57.02 | 6.63 |

### EXAMPLE 15

### 1-(4-CHLOROBUTOXY)-2-[2-(3-METHOXYPHENYL)ETHYL]BENZENE (COMPOUND VI, STEP C, SCHEME B, INTERMEDIATE) SYNTHESIS

A solution of t-BuOH (6 ml) and KOH (276.6 mg; 4.94 mmol) was heated to 80° C. to complete dissolution of KOH. The solution was cooled to room temperature and phenol (563.4 mg; 2.47 mmol) previously dissolved in a minimum amount of t-BuOH was added dropwise thereto. In another flask a solution of 1-bromo-4-chlorobutane (3.70 mmol) in t-BuOH (minimum amount) was prepared and kept under stirring at room temperature. The solution of potassium phenate was added, by dropping funnel, to the solution of 1-bromo-4-chlorobutane in t-BuOH, in an ice bath and under stirring. The mixture was kept under stirring at room temperature for 48 h. Thereafter, t-BuOH was evaporated under reduced pressure and the crude product, recollected with Et₂O, was submitted to five washes with H₂O. The organic phase, dried and filtered, was evaporated under reduced pressure.

Yield: 75.6%

¹H NMR(CDCl₃): δ 1.92-2.08 (m, 4H, CH₂—CH₂); 2.81-2.94 (m, 4H, CH₂CH₂); 3.55-3.69 (m, 2H, CH₂Cl); 3.78 (s, 3H, OCH₃); 3.98-4.03 (m, 2H, OCH₂); 6.73-6.90 (m, 5H, Ar); 7.1-7.21 (m, 3H, Ar) ppm.

MS (m/z): M+ 318 (30); 107, (100)

### EXAMPLE 16

### N-(3-CHLORO)PHENYL-1-(4-{2-[2-(3-METHOXYPHENYL)ETHYL]PHENOXY}BUTYL)-PIPERAZINE (EC77) SYNTHESIS

The aryloxybutyl derivative (200 mg; 0.63 mmol), dissolved in a minimum amount of CH₃CN, was added to a solution of K₂CO₃ (104.33 mg; 0.76 mmol) in CH₃CN (15 ml), kept under stirring at 80° C. Lastly, N-chlorophenylpiperazine (0.63 mmol), previously dissolved in a minimum amount of CH₃CN, was added to the solution. The mixture was kept under stirring at 80° C. for 4 h, under reflux. Thereafter, K₂CO₃ was filtered on a septum and the solid was washed with CH₂Cl₂. The solvent was evaporated under reduced pressure.

The crude product was purified by column chromatography, using acetate/hexane mixture in a 7:3 ratio as eluent, and by subsequent transformation to hydrochloride.

Yield: 16.3%

¹H NMR(CDCl₃): δ 1.90-2.20 (m, 4H, CH₂CH₂); 2.74-2.98 (m, 4H, CH₂CH₂); 3.10-3.30 (m, 2H, CH₂N); 3.41-3.59 (m, 4H, CH₂CH₂ pip.); 3.78 (s, 3H, OCH₃); 3.85-4.09 (m, 4H, CH₂CH₂ pip.); 4.39-4.59 (m, 2H, OCH₂); 6.50-6.96 (m, 5H, Ar); 7.12-7.23 (m, 3H, Ar); 7.34-7.45 (m, 2H, Ar); 7.55-7.65 (m, 1H, Ar); 7.69-7.73 (m, 1H, Ar) ppm.

MS (m/z): M⁺ 478 (34); 249 (26)

mp: 65-73° C.

Elemental Analysis:

| C₂₉H₃₉N₂O₃Cl₂ | N | C | H |
|---|---|---|---|
| Calculated % | 4.92 | 61.11 | 6.85 |
| Found % | 4.95 | 62.79 | 6.89 |

### EXAMPLE 17

### N-(3-TRIFLUOROMETHYL)PHENYL-1-(4-{2-[2-(3-METHOXYPHENYL)ETHYL]PHENOXY}BUTYL)-PIPERAZINE (EC76) SYNTHESIS

The product was obtained by following the procedure described in example 16. The crude product was purified by column chromatography, using acetate/hexane mixture in a 7:3 ratio as eluent, and by subsequent transformation to hydrochloride.

Yield: 26%

¹H NMR(CDCl₃): δ 1.75-1.95 (m, 4H, CH₂CH₂); 2.49 (t, 2H, J=7.0 Hz, CH₂N); 2.58 (t, 4H, J=4.9 Hz, CH₂CH₂ pip.); 2.85-2.95 (m, 4H, CH₂CH₂); 3.21 (t, 4H, J=4.9 Hz, CH₂CH₂ pip.); 3.77 (s, 3H, OCH₃); 4.01 (t, 2H, J=5.8 Hz, OCH₂) 6.72-6.90 (m, 6H, Ar); 7.02-7.38 (m, 6H, Ar) ppm.

MS (m/z): M⁺ 512 (6); 199 (100)

mp: 78-83° C.

Elemental Analysis:

| C₃₀H₃₉N₂O₃F₃Cl₂ | N | C | H |
|---|---|---|---|
| Calculated % | 4.64 | 59.70 | 6.47 |
| Found % | 4.44 | 58.16 | 6.10 |

### EXAMPLE 18

### N-(4-FLUORO)PHENYL-1-(4-{2-[2-(3-METHOXYPHENYL)ETHYL]PHENOXY}BUTYL)-PIPERAZINE (EC78) SYNTHESIS

The product was obtained by following the procedure described in example 16. The crude product was purified by column chromatography, using acetate/hexane mixture in a 7:3 ratio as eluent, and by subsequent transformation to hydrochloride.

Yield: 9.3%

¹H NMR(CDCl₃): δ 1.90-2.20 (m, 4H, CH₂CH₂); 2.81-2.95 (m, 4H, CH₂CH₂); 3.15-3.32 (m, 2H, CH₂N); 3.42-3.59 (m, 4H, CH₂CH₂ pip.); 3.78 (s, 3H, OCH₃); 3.98-4.25 (m, 4H, CH₂CH₂ pip.); 4.73 (m, 2H, OCH₂); 6.65-6.96 (m, 5H, Ar); 7.11-7.23 (m, 6H, Ar); 7.88-8.00 (m, 1H, Ar) ppm.

MS (m/z): M⁺ 462 (13); 205 (27)

mp: 88-95° C.

Elemental Analysis:

| C₂₉H₃₉N₂O₃FCl₂ | N | C | H |
|---|---|---|---|
| Calculated % | 5.06 | 62.93 | 7.05 |
| Found % | 4.26 | 62.22 | 6.65 |

### EXAMPLE 19

### N-(2-METHOXY)PHENYL-1-(4-{2-[2-(3-METHOXYPHENYL)ETHYL]PHENOXY}BUTIL)-PIPERAZINE (EC74) SYNTHESIS

The product was obtained by following the procedure described in example 16. The crude product was purified by column chromatography, using acetate/hexane mixture in a 7:3 ratio as eluent and subsequent transformation to hydrochloride.

Yield: 15.3%

¹H NMR(CDCl₃): δ 1.65-1.95 (m, 4H, CH₂CH₂); 2.50 (t, 2H, J=7.2 Hz, CH₂N); 2.6-2.71 (m, 4H, CH₂CH₂ pip.); 2.73-2.95 (m, 4H, CH₂CH₂); 3.01-3.15 (m, 4H, CH₂CH₂ pip.); 3.78 (s, 3H, OCH₃); 3.86 (s, 3H, OCH₃); 4.01 (t, 2H, J=5.8 Hz, CH₂O); 6.71-7.00 (m, 9H, Ar); 7.09-7.20 (m, 3H, Ar) ppm.

MS (m/z): M⁺ 474 (73); 205 (100)

mp: 100-110° C.

Elemental Analysis:

| C₃₀H₄₀N₂O₃Cl₂ | N | C | H |
|---|---|---|---|
| Calculated % | 5.12 | 65.81 | 7.31 |
| Found % | 5.39 | 66.00 | 7.81 |

### EXAMPLE 20

### N-BOC-3-([2-(2-(PHENYL)ETHYL)PHENOXY]METHYLPIPERIDINE SYNTHESIS (COMPOUND VII, SCHEME B, STEP c, INTERMEDIATE)

N-BOC-3-hydroxymethyl-piperidine (1.00 g, 4.65 mmol) dissolved in a minimum amount of CH₂Cl₂ (5 ml), PPh₃ (1.32 g, 5.03 mmol) and diethyl-aza-decarboxylate (DEAD) (875 mg, 0.80 ml) were added to a solution of phenol IV (3.87 mmol) in CH₂Cl₂ (15.50 ml). The reaction mixture was left under stirring at room temperature for 12 h. Thereafter, the solution was recollected with CH₂Cl₂ and submitted to various washes with small volumes of H₂O and 1N NaOH. The organic phase was dried, filtered and evaporated under reduced pressure, affording the product; the latter was not submitted to further purification but used as such in the subsequent reaction.

¹H NMR (CDCl₃): δ 1.23-1.30 (m, 3H, pip); 1.44 (s, 9H, N-BOC); 1.63-1.83 (m, 4H, pip); 2.79-2.91 (m, 4H, CH₂CH₂); 3.45-3.56 (m, 2H, CH₂—O); 4.15-4.28 (m, 2H, pip); 6.84-6.99 (m, 4H, Ar); 7.22-7.45 (m, 5H, Ar) ppm.

### EXAMPLE 21

### N-BENZYL-3-([2-(2-(3-METHOXYPHENYL)ETHYL)PHENOXY]METHYLPIPERIDINE (R28) SYNTHESIS

KOH (94.08 mg, 1.68 mmol) was added to a solution of benzyl bromide (82.47 mg, 0.48 mmol) in DMSO (1.10 ml) and the mixture was submitted to heating at 40° C. Then, derivative VI (scheme B) (0.48 mmol) dissolved in a minimum amount of DMSO was added. The mixture was left to react at 40° C. for 12 h. Thereafter, the solution was recollected with AcOEt and submitted to repeated washes with H₂O and 1N NaOH. The organic phase was dried, filtered and evaporated under reduced pressure, affording a dark oil.

The crude compound was purified by a further wash with H₂O and 1N NaOH. The organic phase, dried, filtered and evaporated under reduced pressure, afforded an oil.

Yield: 42%

¹H MNR (CDCl₃): δ 1.60-1.80 (m, 7H, pip); 2.62-3.02 (m, 6H, CH₂CH₂+ pip); 3.50 (s, 2H, CH₂O); 3.77 (s, 3H, OCH₃); 3.79-3.87 (m, 2H, CH₂Ph); 6.71-6.84 (m, 4H, Ar); 7.12-7.40 (m, 9H, Ar) ppm.

MS (m/z): M⁺ 415; 83 (98); 91 (100.0).

Elemental Analysis:

| C₂₈H₃₃O₂N | C | H | N |
|---|---|---|---|
| Calculated % | 80.11 | 7.90 | 3.37 |
| Found % | 79.93 | 7.67 | 3.63 |

### EXAMPLE 22

### 2-[2-(2-(3-METHOXY)PHENYLETHYL)PHENOXY]METHYLOXIRANE SYNTHESIS (Scheme C, compound IX, step e, intermediate)

A solution of KOH (150 g, 2.67 mmol) and t-BuOH (3.65 ml) was heated to 80° C., to complete dissolution of KOH; then, the solution was cooled to room temperature and phenol derivative IV (1.32 mmol), dissolved in a minimum amount of t-BuOH, was added dropwise. Concomitantly, in another flask, a solution of epibromidrine (2.67 mmol) in t-BuOH (0.43 mmol) was prepared and stirred at room temperature. The solution of potassium phenate was transferred into a dropping funnel and added dropwise in the epibromidrine solution, kept at 0° C. The mixture was left stirring at room temperature for 19 hours. Thereafter, solvent was evaporated under reduced pressure, and the residue recollected with Et₂O and washed with H₂O. The dried organic phase was evaporated, giving as product an oil that was not submitted to any purification.

Yield: 78%

¹H NMR (CDCl₃): δ 2.77-2.81 (m, 1H, 2H, CH₂₋O); 2.83-2.94 (m, 4H, CH₂CH₂); 3.34-3.41 (m, 1H, CH); 3.78 (s, 3H, OCH₃); 3.98 (dd, 1H, J=−11, 5.3 Hz, CH₂O); 4.22 (dd, 1H, J=−11, 3.1 Hz, CH₂O); 6.76-6.92 (m, 4H, Ar); 7.10-7.78 (m, 4H, Ar) ppm.

MS (m/z): M⁺ 284 (18.24).

Elemental Analysis:

| C₁₈H₂₀O₃ | C | H |
|---|---|---|
| Calculated % | 76.03 | 16.88 |
| Found % | 76.28 | 17.13 |

### EXAMPLE 23

### 1-[1,2,3,4-TETRAHYDROISOQUINOLYL]-3-[2-(2-(3-METHOXY)PHENYLETHYL)PHENOXY]PROPAN-2-OL (MC67) SYNTHESIS

LiClO₄ (1.6 g, 15 mmol) was added to a solution of 2-[2-(2-(3-methoxy)phenylethyl)phenoxy]methyloxirane (425 g, 1.5 mmol) in CH₃CN (3 ml) and the mixture was left under stirring at room temperature for some minutes. Then, tetrahydroisoquinoline (1.5 mmol) dissolved in a minimum amount of CH₃CN was added dropwise. The solution was kept under reflux (T=80° C.) for 2 h. Thereafter, the reaction mixture was recollected with Et₂O and washed with small volumes of a saturated NaCl solution. The organic phase was dried, filtered and evaporated under reduced pressure. The crude compound was purified by transformation to hydrochloride.

Yield: 25%.

mp: 150° C.

¹H NMR (CDCl₃): δ 2.71 (t, 2H, J=6.2, CH₂); 2.77-2.82 (m, 2H, CH₂O); 2.86-2.90 (m, 4H, CH₂CH₂); 2.93 (t, 2H, J=6.2 Hz, CH₂); 3.62-3.70 (m, 3H, CHOH, CH₂); 3.77 (m, 3H, OCH₃); 4.05 (t, 2H, J=5.2 Hz, CH₂N) 6.73-7.24 (m, 12H, Ar) ppm.

Elemental Analysis:

| C₂₇H₃₁NO₃ | C | H | N |
|---|---|---|---|
| Calculated % | 77.67 | 7.48 | 3.35 |
| Found % | 77.70 | 7.49 | 3.39 |

### EXAMPLE 24

### N-[(3-TRIFLUOROMETHYL)PHENYL]-1-(3-{2-[2-(3-METHOXYPHENYL)ETHYL]PHENOXY}PROPAN-2-OL-PIPERAZINE (MC74) SYNTHESIS

LiClO₄ (864,42 mg, 8.125 mmol) dissolved in CH₃CN (3.17 ml, 0.880 mmol) was added to a solution of 3-(trifluoromethyl)piperazine (0.885 mmol), dissolved in a minimum amount of CH₃CN. Then, methyloxirane derivative IX (scheme C) (0.880 mmol) dissolved in a minimum amount of CH₃CN was added dropwise. The mixture was left under stirring at room temperature for 4 h. Thereafter, the solvent was evaporated under reduced pressure, and the residue recollected with Et₂O and washed with small volumes of a saturated NaCl solution. The organic phase was dried, filtered and evaporated under reduced pressure. The crude product was purified by transformation to hydrochloride and crystallized from CH₂Cl₂/Hexane

Yield: 39%

¹H NMR (CDCl₃): δ 2.61-2.71 (m, 4H, piperazine) 2.77-2.82 (m, 2H, CH₂O); 2.85-2.90 (m, 4H, CH₂CH₂); 3.24-3.29 (m, 4H, piperazine); 3.78 (s, 3H, OCH₃); 4.00-4.05 (m, 2H, CH₂N); 4.17-4.18 (m, 1H, CHOH); 6.73-7.36 (m, 12H, Ar) ppm

mp: 130°-140° C.

MS (m/z): M⁺ 514 (47%)

Elemental Analysis:

| C₂₉H₃₃N₂O₃F₃•HCl•½H₂O | C | H | N |
|---|---|---|---|
| Calculated % | 63.27 | 6.18 | 5.09 |
| Found % | 62.90 | 6.08 | 4.80 |

### EXAMPLE 25

### N-[(4-FLUORO)PHENYL]-1-(3-{2-[2-(3-METHOXYPHENYL)ETHYL]PHENOXY}PROPAN-2-OL-PIPERAZINE (MB31) SYNTHESIS

The compound was synthesized as reported in example 24, and was purified by transformation to hydrochloride and crystallization from MeOH

Yield: 22%

¹H NMR (CDCl₃): δ 2.60-2.69 (m, 4H, piperazine); 2.76-2.85 (m, 2H, CH₂O); 2.90-2.95 (m, 4H, CH₂CH₂); 3.05-3.17 (m, 4H, piperazine); 3.76 (s, 3H, OCH₃); 4.00-4.04 (m, 2H, CH₂); 4.13-4.17 (m, 1H, CHOH); 6.73-7.20 (m, 12H, Ar) ppm

MS (m/z): M⁺ 464

### EXAMPLE 26

### N-[(2-METHOXY)PHENYL]-1-(3-{2-[2-(3-METHOXYPHENYL)ETHYL]PHENOXY}PROPAN-2-OL-PIPERAZINE (MB32) SYNTHESIS

The compound was synthesized as reported in example 24, and was purified by transformation to hydrochloride and crystallized from I—PrOH/MeOH

Yield: 15%

¹H NMR (CDCl₃): δ 2.65-2.80 (m, 4H, piperazine) 2.90-2.94 (m, 6H, CH₂CH₂; CH₂O); 3.05-3.11 (m, 4H, piperazine); 3.78 (s, 3H, OCH₃); 3.87 (s, 3H, OCH₃); 4.01-4.04 (m, 2H, CH₂); 4.17 (m, 1H, CHOH); 6.72-7.03 (m, 10H, Ar); 7.11-7.21 (m, 2H, Ar) ppm

MS (m/z): M⁺ 476 (10)

### EXAMPLE 27

### N-[2-PYRIMIDYL]-1-(3-{2-[2-(3-METHOXYPHENYL)ETHYL]PHENOXY}PROPAN-2-OL-PIPERAZINE (MB33) SYNTHESIS

The compound was synthesized as reported in example 24, and was purified by transformation to hydrochloride and crystallized from MeOH.

Yield: 28%

¹H NMR (CDCl₃): δ 2.48-2.80 (m, 4H, piperazine); 2.90-2.94 (m, 6H, CH₂CH₂; CH₂O); 3.77 (s, 3H, OCH₃); 3.83-3.88 (m, 4H, piperazine); 4.00-4.04 (m, 2H, CH₂); 4.14-4.18 (m, 1H, CHOH) 6.50 (t, J=4.8 Hz, 1H5 pyrimidine); 6.70-7.23 (m, 8H, Ar); 8.32 (d, J=4.8 Hz, 2H, H4 and H6 pyrimidine) ppm

MS (m/z): M+ 448

Elemental Analysis:

| C₂₆H₃₂N₂O₃N₄•HCl•½H₂O | C | H | N |
|---|---|---|---|
| Calculated % | 63.15 | 6.88 | 11.33 |
| Found % | 63.32 | 6.63 | 11.11 |

1. A compound having formula: 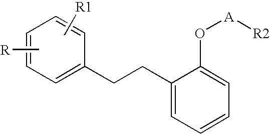
where:
R and R1 may independently be hydrogen atoms, a (C1-C4) alkyl group, a (C1-C3) alkoxy group, a halogen atom, a cyano group, a thiol group, a hydroxyl group, a (C1-C3) thioalkyl group;
R2 may be a 5- or 6-member aryl group, unsubstituted or substituted with a (C1-C3) alkyl group or with a (C1-C3) alkoxy group or with a halogen atom; or a 5- or 6-member aromatic or aliphatic heterocyclic group, containing one or more atoms selected from oxygen, sulphur or nitrogen, also N—(C1-C3)alkyl-, N—(C1-C3)halogenalkyl, N—(C1-C3)alkylamino-substituted or N—(C1-C3)alkyl-aryl-, N—(C1-C3)alkyl-halogenaryl-substituted, N—(C1-C3)alkylaminoaryl-substituted, or it may be an —NR3R4-type group, where R3 and R4 may independently be a hydrogen atom, (C1-C5) alkyl groups, (C1-C5) alkyl groups substituted with 5- or 6-member aromatic or heteroaromatic systems, or (C1-C5) alkyl chains bound to the nitrogen to form a 5- or 6-member aliphatic heterocycle condensed or bound to a further aromatic or heteroaromatic group, also with 5- or 6-members, and
A represents a linear or branched C1-C4 alkyl chain or a single or repeating system of formula —CH₂—CH(OH)CH₂—.

2. The compound according to claim 1, wherein A is a —CH₂— group and R₂ is a phenyl, pyridine or piperidine group, all optionally substituted.

3. The compound according to claim 1, wherein A is a —(CH₂)₂— or —(CH₂)₃— or —CH₂—CH(OH)—CH₂— linear chain and R₂ comprises a piperazine or a tetrahydroisoquinoline group, optionally substituted with other aryl or nitrogenated heterocycle group.

4. The compound according to claim 1 selected from the family consisting of:
1-[(3-methoxybenzyl)oxy]-2-[2-(3-methoxyphenyl)ethyl]benzene (EB27);
2({2-[2-(3-methoxyphenyl)ethyl]phenoxy}methyl]pyridine (EB12); and
3-{2-[2-(3-methoxyphenyl)ethyl]phenoxy}propan-1-[4-(2-pyrimidyl)piperazine] (EB25).

5. A therapeutic treatment of oncological pathologies or central nervous system related pathologies involving P-glycoprotein function comprising administering a therapeutically effective amount of a compound according to claim 1.

6. The therapeutic treatment of claim 5 further comprising administering an anti-tumor medicament or a medicament active on the central nervous system.

7. The therapeutic treatment of claim 5 wherein the compound is selected from: 1-[(3-methoxybenzyl)oxy]-2-[2-(3-methoxyphenyl)ethyl]benzene (EB27);
2({2-[2-(3-methoxyphenyl)ethyl]phenoxy}methyl]pyridine (EB12); and
3-{2-[2-(3-methoxyphenyl)ethyl]phenoxy}propan-1-[4-(2-pyrimidyl)piperazine] (EB25).

8. A pharmaceutical composition comprising a therapeutically effective amount of a compound according to claim 1 and a pharmacologically acceptable excipient.

9. The pharmaceutical composition of claim 8 wherein the compound is selected from the family consisting of:
1-[(3-methoxybenzyl)oxy]-2-[2-(3-methoxyphenyl)ethyl]benzene (EB27);
2([2-[2-(3-methoxyphenyl)ethyl]phenoxy}methyl]pyridine (EB12); and
3-{2-[2-(3-methoxyphenyl)ethyl]phenoxy}propan-1-[4-(2-pyrimidyl)piperazine] (EB25).

10. A pharmaceutical composition comprising the association of a compound according to claim 1 with an anti-tumor medicament or a medicament active on the central nervous system, and a pharmacologically acceptable excipient.

11. The pharmaceutical composition of claim 10 wherein the compound is selected from the family consisting of:
1-[(3-methoxybenzyl)oxy]-2-[2-(3-methoxyphenyl)ethyl]benzene (EB27);
2([2-[2-(3-methoxyphenyl)ethyl]phenoxy}methyl]pyridine (EB12); and
3-{2-[2-(3-methoxyphenyl)ethyl]phenoxy}propan-1-[4-(2-pyrimidyl)piperazine] (EB25).

12. A pharmaceutical kit comprising a first element containing a compound according to claim 1 and a second element containing an anti-tumor medicament or a medicament active on the central nervous system, for combined, concomitant or sequential use.

13. The pharmaceutical kit of claim 12 wherein the compound is selected from the family consisting of:
1-[(3-methoxybenzyl)oxy]-2-[2-(3-methoxyphenyl)ethyl]benzene (EB27);
2([2-[2-(3-methoxyphenyl)ethyl]phenoxy}methyl]pyridine (EB12); and
3-{2-[2-(3-methoxyphenyl)ethyl]phenoxy}propan-1-[4-(2-pyrimidyl)piperazine] (EB25).

14. A method of preparing a compound according to claim 1 comprising the steps of:
reacting a compound of formula IV according to the following scheme (scheme B); 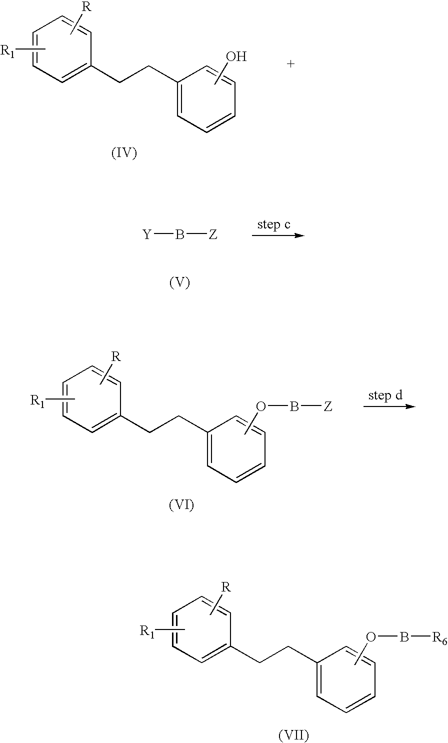
isolating the obtained compound; and
optionally purifying it.

15. A method of preparing the compounds according to claim 1 comprising the steps of:
reacting a compound of formula IV according to the following scheme (scheme C); 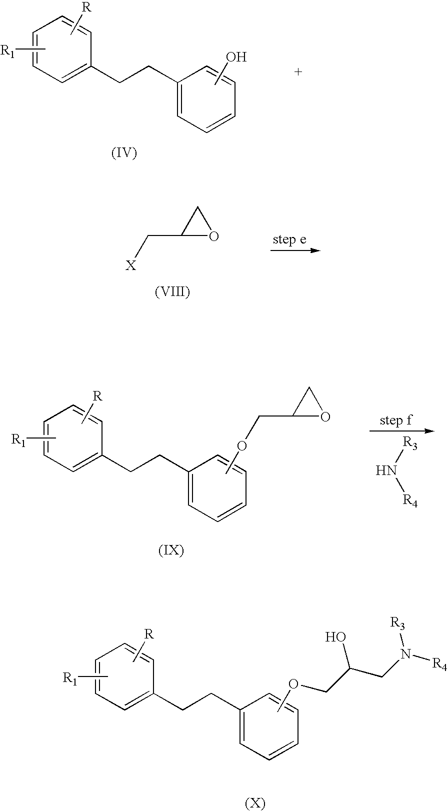
isolating the obtained compound; and
optionally purifying it.

16. A method of preparing the compounds according to claim 1 comprising the steps of:
reacting a compound of formula IV according to the following scheme (scheme D); 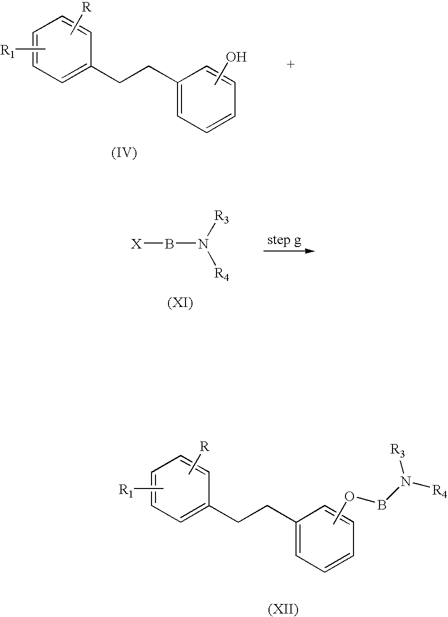
isolating the obtained compound; and
optionally purifying it.

17. A method of preparing the compounds according to claim 1 comprising the steps of:
reacting a compound of formula IV according to the following scheme (scheme E); 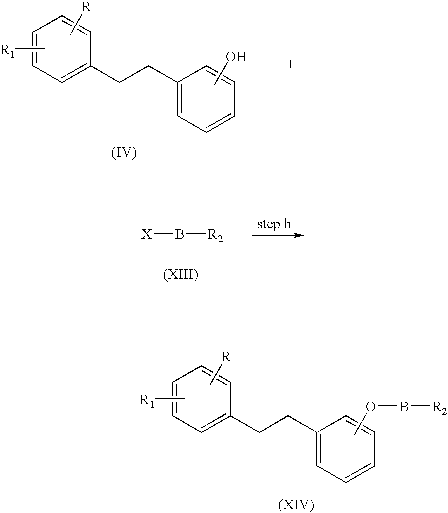
isolating the obtained compound; and
optionally purifying it.
